(19) United States
(12) Patent Application Publication
(10) Pub. No.: US 2011/0029050 A1
(43) Pub. Date: Feb. 3, 2011

(54) INTRA-VENTRICULAR BRAIN COOLING CATHETER

(76) Inventors: John Elefteriades, Guilford, CT (US); Grahame Gould, Hander, CT (US); Remo Moomiae-Qajar, Providence, RI (US); John W. Simmons, Woodbury, CT (US); Eric Butt, Milford, CT (US); Joel N. Helfer, Cheshire, CT (US); Jeffrey E. Ransden, Fairfield, CT (US)

Correspondence Address: WELSH FLAXMAN & GITLER LLC, 2000 DUKE STREET, SUITE 100, ALEXANDRIA, VA, 22314, US

(21) Appl. No.: 12/621,034

(22) Filed: Nov. 18, 2009

Related U.S. Application Data
(60) Provisional application No. 61/115,712, filed on Nov. 18, 2008.

(51) Int. Cl. A61F 7/12

(52) U.S. Cl. 607/105

(57) ABSTRACT

A method for cooling of a brain with localized hypothermia allowing for maintenance of the core body temperature is achieved by positioning a cooling catheter within a ventricular cavity of the brain. The cooling catheter includes an inlet channel and an outlet channel providing for a closed flow of a cooling fluid into and out of the cooling catheter. A sack is formed at a distal end of the cooling catheter. The sack is in fluid communication with distal ends of the inlet channel and the outlet channel such that the sack is continually flushed with the cooling fluid as the cooling fluid flows into and out of the cooling catheter. The sack, when filled, takes the shape and size of the ventricular cavity filling the ventricular cavity in which it is positioned. The method further includes cooling the cooling catheter and the ventricular cavity through the closed flow of the cooling fluid through the cooling catheter.

### CROSS REFERENCE TO RELATED APPLICATION

This application claims the benefit of U.S. Provisional Patent Application Ser. No. 61/115,712, entitled “INTRA-VENTRICULAR BRAIN COOLING CATHETER”, filed Nov. 18, 2008.

### BACKGROUND OF THE INVENTION

1. Field of the Invention

The present invention relates to an apparatus and associated method for cooling the brain. In particular, the invention relates to an intra-ventricular catheter and associated method for cooling the brain via positioning of a cooling sack within the ventricular space of the brain.

2. Description of the Prior Art

Despite advances in spinal cord protection, paraplegia continues to be a serious complication of descending and thoracoabdominal aortic operations. Paraplegia has been a serious and vexing problem since the advent of direct thoracic aortic surgery some 40 years ago. Paraplegia continues to devastate the lives of patients undergoing surgery for thoracic aortic aneurysm; in cases of post-operative paraplegia, mortality is high and, even in survivors, quality of life is devastated.

Spinal ischemia is a known postoperative complication following aortic surgeries. The incidence of spinal cord ischemia during aortic surgery is typically over 10%. During thoracic or thoracoabdominal aortic aneurysm repair, for example, the spinal arteries, which provide blood supply to the spinal cord, are often severed from the diseased aorta, and some but not all are later resutured to a prosthetic graft. As a result, blood flow to the spinal cord is reduced. When aortic clamp time and consequent reduction of spinal perfusion lasts more than 45 minutes, spinal ischemia ensues, often resulting in paralysis.

In recent years, there is a general sense that improvements are being made in better preventing paraplegia. Multiple advances have expanded the anti-paraplegia armamentarium. Re-discovery of left atrial-to-femoral artery perfusion for descending and thoracoabdominal operations permits reliable perfusion of the lower body and spinal cord. Collagen-impregnated grafts have improved hemostasis and inherent handling characteristics of available prostheses. Identification and re-implantation of spinal cord arteries has improved. Spinal cord drainage, aimed at improving the perfusion gradient for the spinal cord by minimizing external pressure on cord tissue, has been adopted almost universally. The advent of effective anti-fibrinolytic agents has decreased peri-operative blood loss and, consequently, led to improved hemodynamics. The importance of maintaining proximal hypertension during the cross-clamp time has been recognized. The fact that that nitroprusside administration is contra-indicated during surgery, because its administration can lead to increased intra-thecal pressure, has also been recognized. In addition, it has been found that by keeping blood pressure high after aortic replacement during the ICU and step-down unit states it is possible to prevent many cases of paraplegia. It has also been found that early recognition and treatment of late post-operative paraplegia can often lead to restoration of spinal cord function; important measures include raising the blood pressure with inotropic medications and volume administration, optimization of hematocrit with blood transfusions, and re-institution of spinal cord drainage.

Yet, with all of the advances described above, and the many more advances not described herein, paraplegia has not been reduced to zero incidence. This continues to be a major issue, both clinically and medico-legally.

Cooling is known to be protective against ischemia for all body tissues, especially the brain and spinal cord. In fact, one group uses instillation of cold fluid into the intra-thecal space to produce core cooling and protect the spinal cord during aortic surgery. Cambria R P, Davison J K, Zannetti S, et al: Clinical experience with epidural cooling for spinal cord protection during thoracic and thoracoabdominal aneurysm repair, J Vasc Surg 25:234-243, 1997. Despite good local results, this technique has not been generally adopted because of concerns about the cumbersome nature of instilling and draining fluid, and because of documented elevation in intra-thecal pressure consequent upon fluid instillation.

The experience of Kouchoukos and colleagues with the performance of descending and thoracoabdominal replacement under deep hypothermic arrest—with a near zero paraplegia rate—demonstrates vividly the powerful protective influence of hypothermia. Yet, most aortic surgeons do not utilize deep hypothermic arrest for descending and thoracoabdominal operations, out of concern for potential negative effects of deep hypothermia and prolonged perfusion in this setting.

It is also known that brain damage associated with either stroke or head trauma is worsened by hyperthermia and improved with hypothermia. As such, and as with the hypothermia treatments for the spinal canal discussed above, various researchers have attempted to utilize hypothermia in treating stroke and head trauma. However, these attempts have met with only limited success.

Of particular relevance is U.S. Pat. No. 6,699,269 to Khanna. This patent provides a method and apparatus for performing selective hypothermia to the brain and spinal cord without the need for systemic cooling. In accordance with the disclosed embodiment, a flexible catheter with a distal heat exchanger is inserted into the cerebral lateral ventricle or spinal subdural space. The catheter generally includes a heat transfer element and three lumens. Two lumens of the catheter circulate a coolant and communicate at the distal heat transfer element for transfer of heat from the cerebrospinal fluid. The third lumen of the catheter allows for drainage of the cerebral spinal fluid.

While the system disclosed in the Khanna patent generally discloses a system for spinal cord and brain cooling, Khanna offers very few details regarding the specific structures and procedures for achieving the goal of spinal cord and brain cooling. As those skilled in the art will certainly appreciate, cooling of the spinal cord or brain is not merely a matter of inserting a catheter having a heat exchanger at a distal end thereof within the space desired for cooling and hoping for the best results. Rather, detailed analysis is required so that such a system may actually function to serve the needs of patients. Khanna fails to provide the specificity required for achieving this goal. As such, Khanna may be considered in much the same category as the other prior art references as not providing a system for sufficiently addressing the goal of spinal cord and brain cooling.

As such, a need exists for a method and apparatus whereby the brain of an individual may be cooled with the hopes of reducing and eliminating injuries. The present invention provides such a method and apparatus.

### SUMMARY OF THE INVENTION

It is, therefore, an object of the present invention to provide a method for cooling of a brain with localized hypothermia allowing for maintenance of the core body temperature. The method is achieved by positioning a cooling catheter within a ventricular cavity of the brain. The cooling catheter includes an inlet channel and an outlet channel providing for a closed flow of a cooling fluid into and out of the cooling catheter. A sack is formed at a distal end of the cooling catheter. The sack is in fluid communication with distal ends of the inlet channel and the outlet channel such that the sack is continually flushed with the cooling fluid as the cooling fluid flows into and out of the cooling catheter. The sack, when filled, takes the shape and size of the ventricular cavity filling the ventricular cavity in which it is positioned. The method further includes cooling the cooling catheter and the ventricular cavity through the closed flow of the cooling fluid through the cooling catheter.

It is also an object of the present invention to provide a method wherein the cooling catheter is a tri-lumen catheter.

It is another object of the present invention to provide a method wherein the cooling catheter is a tri-lumen catheter and, in addition to the inlet channel and the outlet channel, includes a stylet channel or fluid drainage channel.

It is a further object of the present invention to provide a method further including the step of drawing fluid from the ventricular cavity via the fluid drainage channel.

It is also an object of the present invention to provide a method wherein the sack is made from a medical grade elastomeric polymer.

It is another object of the present invention to provide a method wherein the step of cooling includes the cooling fluid flowing down the inlet channel, into the sack, and back up the outlet channel, providing for filling and expansion of the sack along with cooling at a location of the sack.

It is a further object of the present invention to provide a method wherein the ventricular cavity is that of a lateral ventricle of the brain.

It is also an object of the present invention to provide a method wherein the step of positioning includes accessing the ventricular cavity via a burr hole.

It is another object of the present invention to provide a method wherein the step of cooling includes cooling the cerebral spinal fluid in the ventricular cavity to a temperature of at between approximately 28° C. and approximately 34° C.

It is a further object of the present invention to provide a method wherein the step of cooling includes cooling for approximately several hours to 3 days.

It is also an object of the present invention to provide a method wherein the cooling catheter includes a monitor measuring intracranial pressure.

It is another object of the present invention to provide a method wherein the cooling catheter includes a ventricular drain.

Other objects and advantages of the present invention will become apparent from the following detailed description when viewed in conjunction with the accompanying drawings, which set forth certain embodiments of the invention.

### BRIEF DESCRIPTION OF THE DRAWINGS

FIGS. 1, 2 and 3 are respectively a top plan view, an exploded side plan view (with the sack not shown) and a cross sectional view of the cooling catheter in accordance with the present invention.

FIGS. 4, 5 and 6 are schematics of alternate systems in accordance with the present invention.

FIGS. 7, 8, 9 and 10 are schematics showing cooling of the brain in accordance with the present invention.

FIG. 11 is a graph presenting test data in accordance with the present invention.

FIG. 12 is a perspective view in accordance with an alternate embodiment of the present invention.

FIGS. 13, 14, 15 and 16 are schematics showing cooling of the brain in accordance with the present invention.

### DESCRIPTION OF THE PREFERRED EMBODIMENTS

The detailed embodiments of the present invention are disclosed herein. It should be understood, however, that the disclosed embodiments are merely exemplary of the invention, which may be embodied in various forms. Therefore, the details disclosed herein are not to be interpreted as limiting, but merely as the basis for the claims and as a basis for teaching one skilled in the art how to make and/or use the invention.

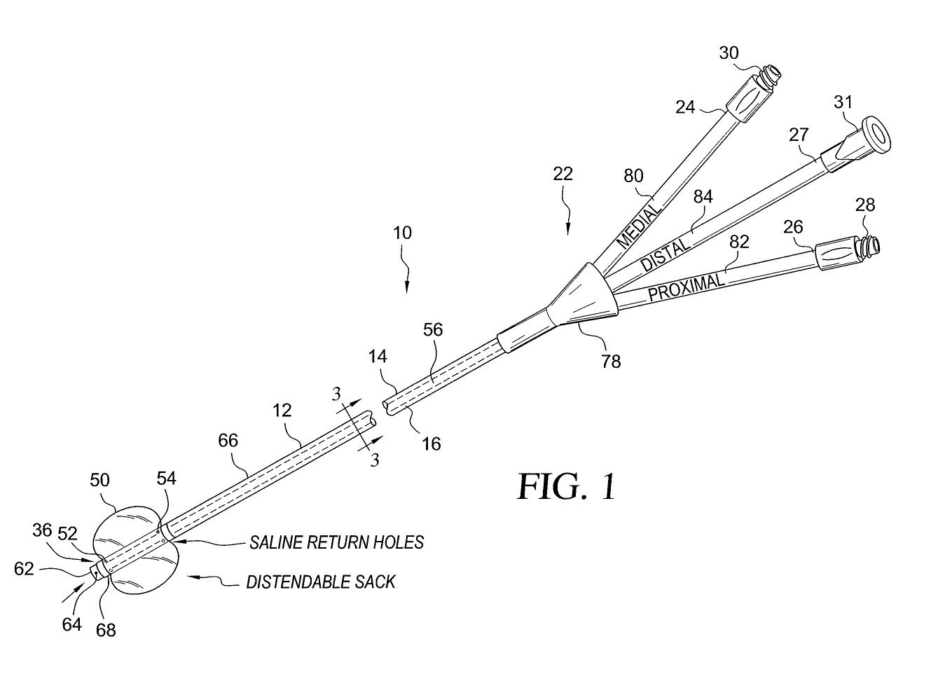

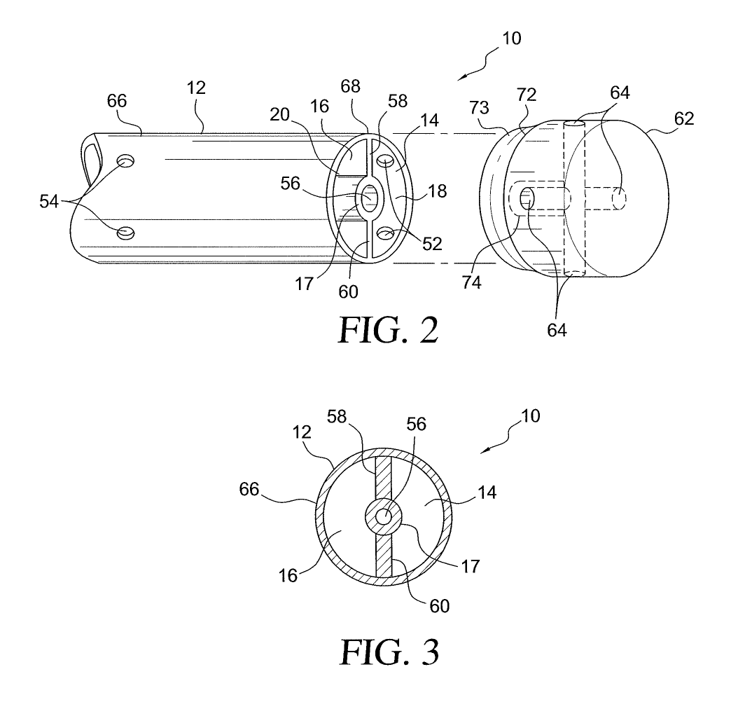

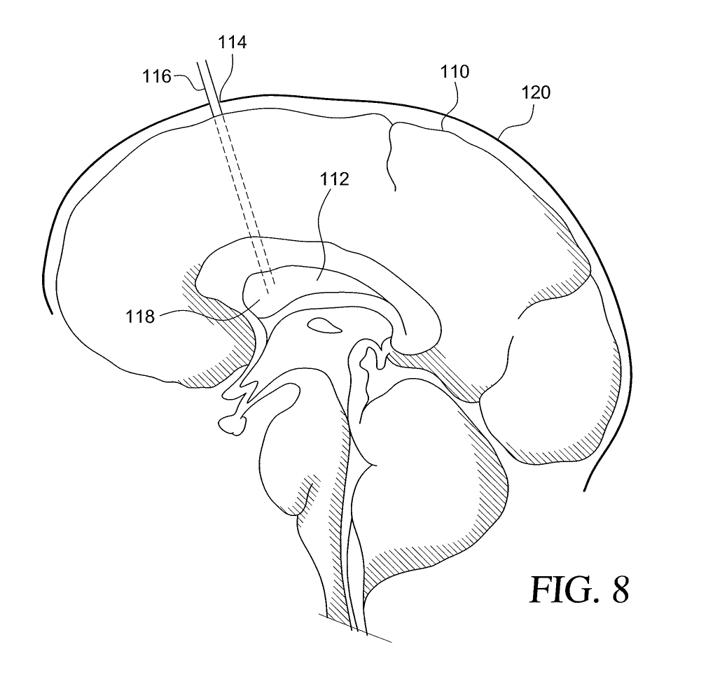

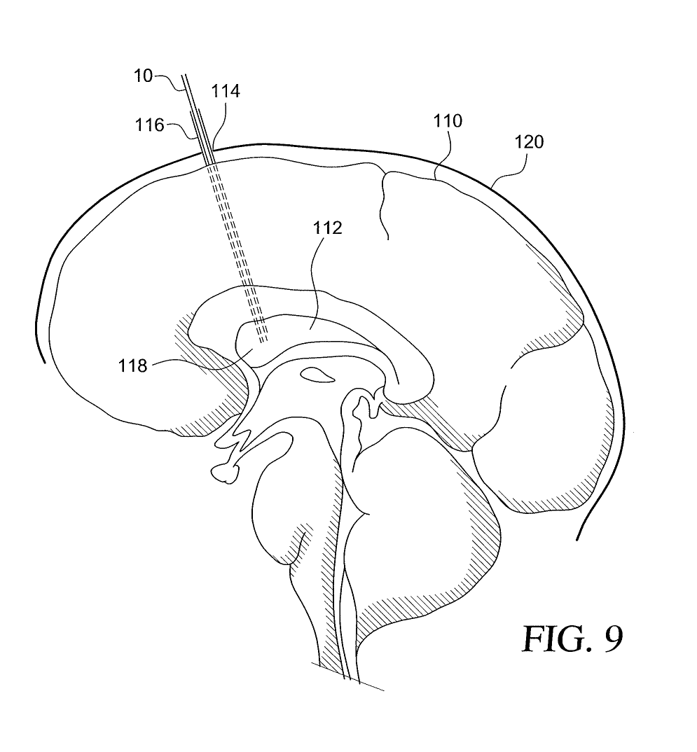

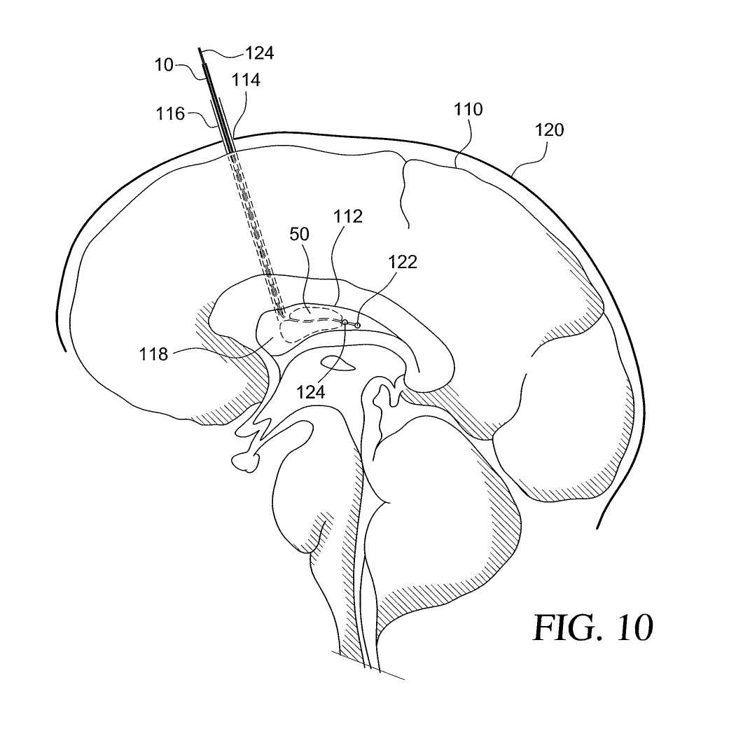

With reference to FIGS. 1 to 10, an intra-ventricular cooling catheter 10 and associated method for cooling of the brain are disclosed. The cooling catheter 10 and method provide an effective mechanism for cooling the brain in an effort to reduce and treat brain injuries. It is contemplated the present cooling catheter 10 may be used during aortic surgery performed with deep hypotheremic circulatory arrest (DHCA), during brain surgery, in the treatment of traumatic brain injuries, in the treatment of ischemic or hemorrhagic stroke. Generally, the present intra-ventricular cooling catheter system 1 includes a closed-loop, cooling catheter 10 coupled to a cooling system 11.

With regard to the intra-ventricular cooling catheter 10 of the present invention, it is a tri-lumen polyurethane catheter with an equal split. That is, the cooling catheter 10 is generally composed of a cylindrical, extruded tube 12 with two hollow semi-circular channels along the interior wall, that is, inlet and outlet channels 14, 16, providing for the flow of cooling fluid into and out of the cooling catheter 10. A center third channel (or fluid drainage channel) 56 is centrally positioned in the middle of the cooling catheter 10 to provide a passageway to drain fluid from the body. As will be appreciated based upon the following disclosure, the disclosed configuration of the channels (or lumens) 14, 16, 56 is an important feature of the cooling catheter 10. As described above, the cooling catheter 10 employs a coaxial design including a center third channel 56 running directly down the middle of the cooling catheter 10 and being held in place by thin walls 58, 60 on either side that effectively divide the outer lumen into two parts, that is, the inlet channel 14 and outlet channel 16. This design allows the channels 14, 16, 56 to be completely separated from one another. The center third channel 56 is to be used as a drain line; the inlet channel 14 and the outlet channel 16 are used to transport chilled saline in and out of a distendable sack 50 at the distal end 36 of the cooling catheter 10.

The cooling catheter 10 has a unique drain tip 62 which fits in the cooling catheter 10 at the distal end 36 thereof. In particular, the catheter body 66 of the cooling catheter 10 is an extruded tubular member and the drain tip 62 is secured to the distal end 68 of the catheter body 66. Secure attachment is preferably achieved through the provision of a projection 73 along the proximal end 72 of the drain tip 62 which seats within one of the channels, for example, the inlet channel 14, for frictional engagement and proper relative orientation therebetween. Secure attachment is further facilitated by the provision of adhesive between the drain tip 62 and the catheter body 66. Since the outer diameter of the drain tip 62 matches the outer diameter of the catheter body 66, a smooth outer profile is achieved when the two components are attached.

The drain tip 62 is a molded polymer piece provided at the distal end 36 of the cooling catheter 10, in particular, attached at the distal end 68 of the catheter body 66. The drain tip 62 has four circumferentially oriented, and evenly spaced, holes 64 formed in a sidewall thereof. The holes 64 communicate with the center third channel 56 via a central fluid channel 74 formed in the drain tip 62 and allow for the drainage of fluids from the body. As will be appreciated based upon the following disclosure, the positioning of the four holes 64 on the sides of the drain tip 62 prevents the blockage of fluid flow as the drain tip 62 comes into contact with wall tissue of the lateral ventricles. Additionally, the drain tip 62 enhances the navigation of the cooling catheter 10 by enabling the surgeon to visually confirm the proper placement of the cooling catheter 10 in the lateral ventricles by allowing the backflow of cerebral spinal fluid through the open fluid drainage channel (that is, the central third channel) 56 to the proximal end 22 of the cooling catheter 10 at the exterior of the body. This navigation and placement feature assures that the self-expanding sack 50 (discussed below in greater detail) is accurately positioned in the lateral ventricle to effect cooling of the cerebral spinal fluid.

More particularly, and in accordance with a preferred embodiment of the present invention, the cooling catheter 10 is approximately 33 cm long. The cooling catheter 10 has an outer diameter of approximately 3.3 min, an inner diameter of approximately 2.7 mm and wall thickness of approximately 50 μm. The septums 17 separating the inlet and outlet channels 14, 16 and the central fluid drainage channel 56 are approximately 50 μm thick.

The distal ends 18, 20 of the inlet and outlet channels 14, 16 formed within the cooling catheter 10 are in fluid communication so that a cooling fluid may be freely circulated within a closed loop extending through the cooling catheter 10. With this in mind, a distendable self-expanding, soft sack 50 is formed at the distal end 36 of the cooling catheter 10. In accordance with a preferred embodiment, the sack 50 is made from a medical grade elastomeric polymer. The sack 50 is shaped and dimensioned such that when it is filled it takes the shape and size of the lateral ventricle 112 in which it is positioned in the manner discussed below in greater detail. The sack 50 is in fluid communication with both the inlet channel 14 and the outlet channel 16 via respective ports 52, 54 allowing for fluid communication between the sack 50 and the respective inlet and outlet channels 14, 16. As a result, the sack 50 is continually flushed with cooling fluid as the cooling fluid moves through the system 1 of the present invention.

More particularly, the sack 50 is a torroidal shape that when inflated will be in the same plane as the end of the drain tip 62 formed at the distal end 36 of the cooling catheter 10. This is significant because it allows the sack 50 maximum penetration into the ventricular space before the cooling catheter 10 bottoms out. As discussed above, the drain tip 62 is a device that has four holes (or drain ports) 64 in a vertical sidewall thereof and each hole 64 communicates with the center third channel (or drain lumen) 56 of the cooling catheter 10. The significance of this layout is that when the cooling catheter 10 is in the ventricular space as discussed below the drain ports 64 on the drain tip 62 will not be occluded as it would be if the center third channel 56 terminated at the bottom of the cooling catheter without the tip in place. This drain tip 62 also acts as a seal for the remaining inlet channel 14 and the outlet channel 16.

The hole locations for the ports 52, 54 on the cooling catheter 10 that fill and drain the sack 50 are also important to its function. There are four ports total, two ports 52 that supply the sack 50 and two ports 54 that drain the sack 50. These two sets of ports 52, 54 are on opposite sides of the cooling catheter 10 and the ports 52 at the distal end of the sack 50 are the supply ports (that is connected to the inlet channel 14) while the ports 54 at the proximal end of the sack 50 are the drain ports 54 (that is connected to the outlet channel 16). This is important because the location of these ports 52, 54 allows the sack 50 to fill properly without trapping air. Each pair of ports 52, 54 communicates respectively with the inlet channel 14 and the outlet channel 16 for the controlled flow of cooling fluid through the sack 50.

There is a manifold 78 on the cooling catheter 10 that has three supply tubes 80, 82, 84 connected to the catheter channels 14, 16, 56. The supply tube 80 feeding the distal ports 52 (that is, via the inlet channel 14) is labeled MEDIAL, the return ports 54 (that is, via the outlet channel 16) communicate with the supply tube 82 labeled PROXIMAL and the drain lumen (that is, the center third channel 56 that communicates with drain ports 64 in the drain tip 62) communicates with the supply tube 84 is labeled DISTAL.

In practice, cooling fluid flows down the inlet channel 14, into the sack 50, and back up the outlet channel 16, providing for filling and expansion of the sack 50 along with cooling at the location of the sack 50 and along the entire length of the cooling catheter 10. At the proximal end 22 of the cooling catheter 10, the inlet and outlet channels 14, 16 split into the individual supply tubes 80, 82. The proximal ends 24, 26 of the respective supply tubes 80, 82 are provided with a luer connection 30, 28 for fitting tubes 32, 34 to supply (inlet) and remove (outlet) cooling fluid from the cooling catheter 10. Similarly, the proximal end 27 of the supply tube 84 includes a connection member 31.

In accordance with a preferred embodiment of the present invention, the cooling catheter 10 is no greater than 10 (3.3 mm) to 13 (4.3 mm) French catheter scale size and is a flexible, atraumatic cooling catheter. In accordance with a preferred embodiment, the catheter is 11.5 French. As the catheter is intended to extend the complete length into the ventricular space of the brain, the catheter will have a length of approximately 33 cm to provide ample catheter length for use during the procedure described below in greater detail. While specific parameters regarding the length and diameter of the catheter are presented herein in accordance with describing a preferred embodiment of the present invention, those skilled in the art will appreciate that these parameters may be varied to suit specific applications without departing from the spirit of the present invention.

With the catheter structure described above in mind, the present cooling catheter 10 is well suited for neurosurgical burr hole approach for placement through the brain into the lateral ventricles. As will be described below in greater detail, burr hole placement of the present cooling catheter 10 adds to the enhanced functionality of the present invention which results in a device specifically suited for cooling the brain.

With regard to the cooling system 11 providing the cooling fluid to the cooling catheter 10, a coolant fluid source 40 supplies coolant fluid to the catheter while maintaining the temperature of the coolant fluid at a predetermined temperature. For example, and in accordance with a preferred embodiment of the present invention, the coolant fluid is maintained at a temperature of −10° C. and is generally composed of an ice and a supersaturated salt solution stored within an insulated container 42. With regard to the cooling fluid that has passed through the cooling catheter 10, it is collected and either re-circulated through the cooling source and into the lateral ventricles or collected with an outlet collection tank 44. Tubing 32, 34 is provided for selective connection to the inlet channel 14, outlet channel 16, coolant fluid source 40 and outlet collection tank 44. The tubing 32, 34 is insulated to minimize thermal loss prior to passage of the coolant fluid within the catheter.

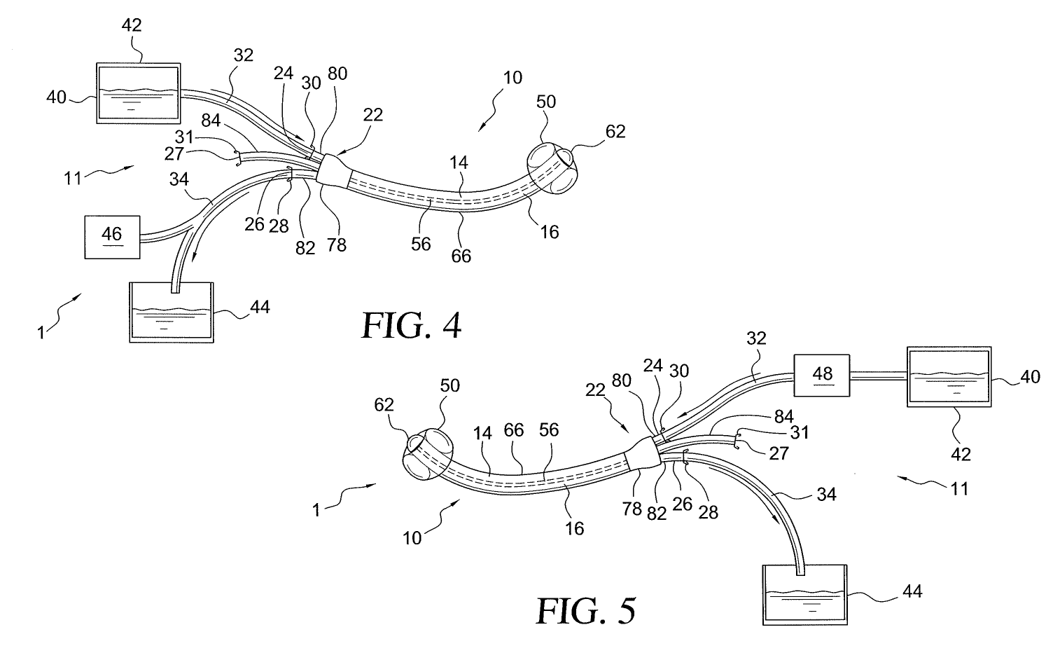

In accordance with preferred embodiments, three variations on a cooling system 11 are contemplated for achieving fluid circulation. In accordance with a first embodiment, and with reference to FIG. 4, the coolant fluid will flow under a vacuum. In particular, the coolant fluid is drawn through the inlet and outlet channels 14, 16 via negative pressure bias. The vacuum 46 is applied to the outlet channel 16. The inlet tubing 32 (in the coolant fluid source 40) has a weighted filter element (not shown) to prevent flow blockages.

In accordance with an alternate embodiment, and with reference to FIG. 5, the coolant fluid flows under positive pressure from a pump 48. In particular, the coolant fluid is pushed through the inlet and outlet channels 14, 16 via positive pressure bias from a pump 48. As with the earlier embodiment, the inlet tubing 32 (in the coolant fluid source 40) has a weighted filter element (not shown) to prevent flow blockages. The pump 48 may be inside or outside of the coolant fluid source depending on specific requirements.

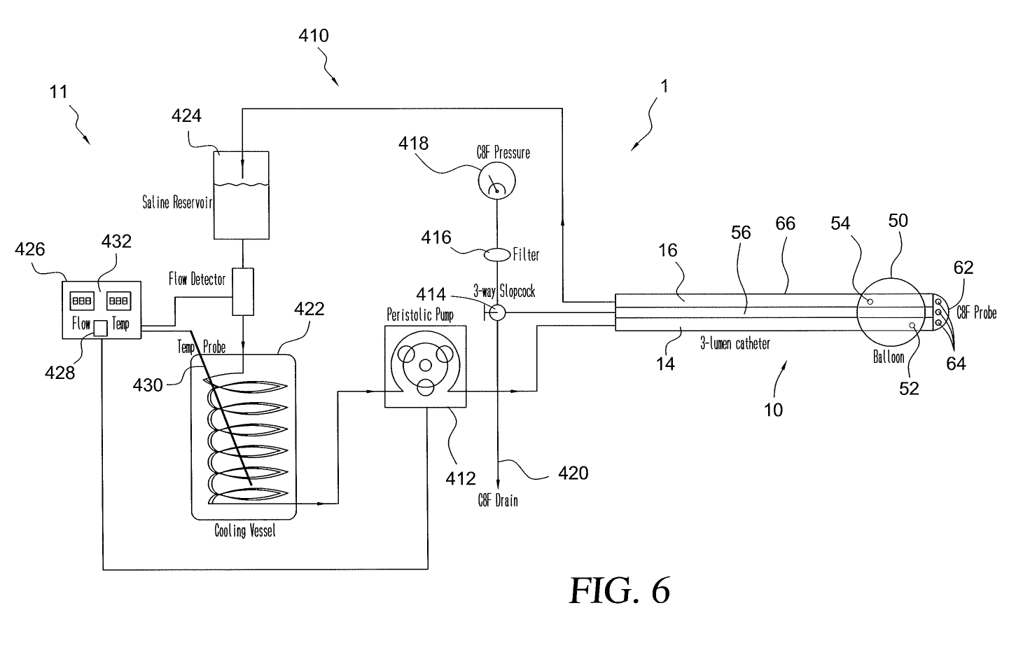

In accordance with another embodiment for creating flow of the cooling fluid, and with reference to FIG. 6, the cooling system 11 includes a fluid circulation system 410 that acts as a controller mechanism. This fluid circulation system 410 preferably includes a micro-pump 412 (for example, a peristaltic pump) to manage the flow. The micro-pump 412 is connected to the cooling catheter 10 via a three-way stopcock valve 414 for connection to the inlet channel 14, outlet channel 16 and center third channel 56. The stopcock valve 414 is connected to a filter 416 leading to a cerebral spinal fluid pressure gauge 418, as well as a drain 420 for the cerebral spinal fluid. The fluid circulation system 410 further includes a chiller device 422 for cooling the fluid as it flows to the inlet channel 14 and back through the outlet channel 16 and into the reservoir 424 in fluid communication with the chiller device 422. The fluid circulation system 410 is also provided with a controller 426 including control software 428 for controlling the entire unit. The controller 426 is further provided with a display 432 showing the flow and temperature data, an interface allowing the operator to program and control the flow and thermisters 430 to monitor temperature. This entire fluid circulation system 410 is preferably housed in a mechanism the size of an IV pump device which hangs on an IV pole and would, therefore, be portable for transport with the patient as he or she is moved to a post-operative area. It is contemplated this will probably be simplified to have frozen slurry in a vessel that is contained within an outer freezer. The circulating saline solution will be drawn from the bottom of the vessel and returned to the vessel that is within the freezer.

Although various cooling systems have been described above for use in accordance with the present invention, it is contemplated other cooling systems could be employed without departing from the spirit of the present invention.

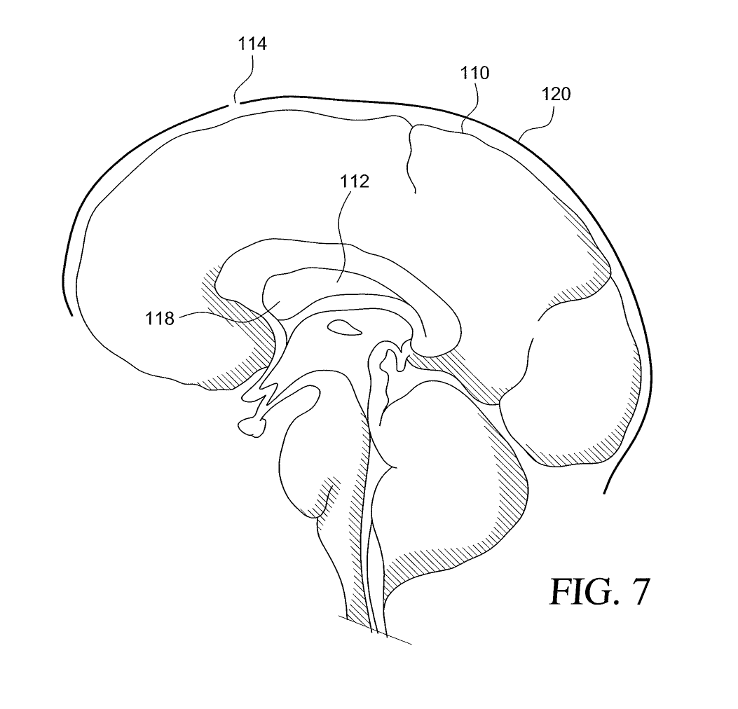

Referring to FIGS. 7 to 10, the present cooling catheter 10 is designed to provide neurologic brain protection against ischemia by inducing moderate hypothermia. Such brain protection would be provided in situations of cerebrovascular accident (for example, stroke) and traumatic brain injuries. In such situations, it is a standard neurosurgical practice to access one lateral ventricle 112 of the brain 110 via a burr hole 114 and a directed needle 116 puncture. As those skilled in the art will certainly appreciate, the lateral ventricles 112 form a portion of the ventricular system of the brain 110 and contain a reservoir of cerebral spinal fluid. In particular, the lateral ventricles 112 connect to the central third ventricle through the interventricular foramina of Monro.

In accordance with a preferred embodiment of the present invention, and with reference to FIGS. 7 to 10, a burr hole 114 is first formed in the skull 120 in accordance with traditional medical procedures those skilled in the art will certainly appreciate. The lateral ventricle 112 is then accessed via the burr hole 114 and the directed needle 116 puncture, the present cooling catheter 10 is inserted through the needle 116 and into the ventricular cavity 118. For use in accordance with this procedure, the cooling catheter 10 is shaped and dimensioned such that the sack 50 may be positioned within the ventricular cavity 118 and then expanded to fill the ventricular cavity 118 when the cooling fluid is pumped therethrough. Once the cooling catheter 10 is properly positioned, cooling fluid is recirculated through the lumens of the cooling catheter 10 as described above. This will cause the sack 50 to fill with cooling fluid, expand and fill the ventricular cavity 118. In general, and as discussed above with regard to the spinal cord applications, the cerebral spinal fluid in ventricular cavity 118 is preferably cooled to a temperature of between approximately 28° C. and approximately 34° C., and maintained at this temperature for between several hours and 1 to 3 days as required.

In this way, the present procedure “spot or locally cools” within the lateral ventricle 112 where cerebral spinal fluid is first encountered after passing through the grey and white matter of the brain. As such, cerebral spinal fluid is cooled, thus cooling the brain as well where it circulates. By cooling the brain, protection is provided since it is well known that hypothermia of even modest proportions (even fractions of a degree) is highly brain protective. Through the utilization of this technique, a brain may be protected against cerebral ischemia in cases of stroke or trauma. In cooling the tissue and organs of the brain that come into contact with the cooled cerebral spinal fluid, the localized or spot cooling of this invention induces therapeutic hypothermia only to the targeted organs. In doing so, this invention allow for systemic body (core) temperature to be maintained while localized hypothermia is induced. This prevents the negative outcomes for systemic cooling such as arrhythmias, chronic shivering, and pneumonia.

Improved functionality of the cooling catheter 10 in the performance of this procedure may be achieved by incorporating a monitor, for example, a pressure transducer 122, for measuring intracranial pressure and a ventricular drain 124 to release intracranial pressure when necessary by draining cerebral spinal fluid.

With this in mind, testing has been performed by the inventors to determine if use of the present cooling catheter within the lateral ventricles of the brain can effectively cool the cerebral spinal fluid (CSF) and thereby reduce brain temperature while maintaining systemic normotherinia. In particular, it is unknown whether a cooling system can overcome the warming by the native cerebral blood flow.

In accordance with the goals of the present study, the present cooling system and cooling catheter were employed to circulate a cold fluid and cool the CSF that circulates in the brain. The CSF in turn cools the surrounding brain by conduction. As discussed above, the cooling catheter is specifically designed for application to the lateral ventricles of the brain. Burr holes were made in the skull and the cooling catheter was placed into the lateral ventricles using the standard method for placement of ventriculostomy catheter. The study was conducted in sheep because their body mass is similar to adult humans. To monitor the cooling effect, four temperature probes were placed in the brain (left and right hemispheres of the brain in anterior and posterior locations to the ventricles).

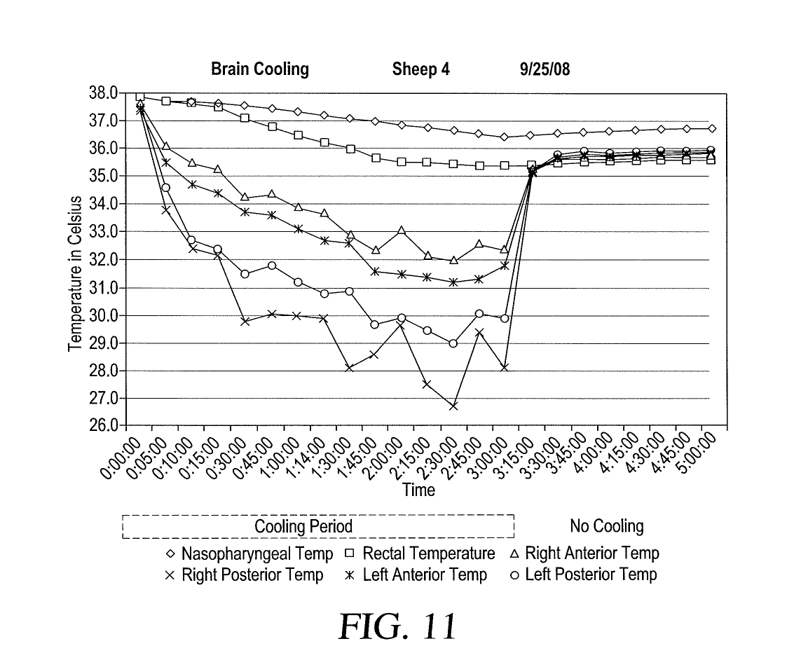

Five experiments were successfully completed (temperature probes modified after first experiment). In each animal, two cooling catheters were successfully placed into the lateral ventricles. The mean brain temperature for all sheep decreased to 34.5° C. (mean) during the 3 hour cooling period. This represented a 9.7% reduction from the average baseline brain temperature of 38.2° C. During the cooling period, the cooling fluid was circulated through the catheter at a maximum rate of 50 ml per minute. The lowest achieved brain temperature during cooling was 26.7° C., which represented a 28.6% decrease from baseline. When cooling was stopped, the brain temperature readings equilibrated with the core temperature promptly. Post-mortem examination of the brains showed no morphologic changes under gross or histologic examinations. Results for Sheep #4 are shown with reference to FIG. 11.

Based upon the results of this study, it has been concluded localized cooling of the brain to moderate hypothermic levels while maintaining relative systemic normothermia was demonstrated in an animal model with the present intraventricular cooling catheters. This technique holds promise as an additional neuroprotection modality to mitigate brain injury in deep hypothermic circulatory arrest for aortic arch surgery as well as in traumatic brain injury and stroke.

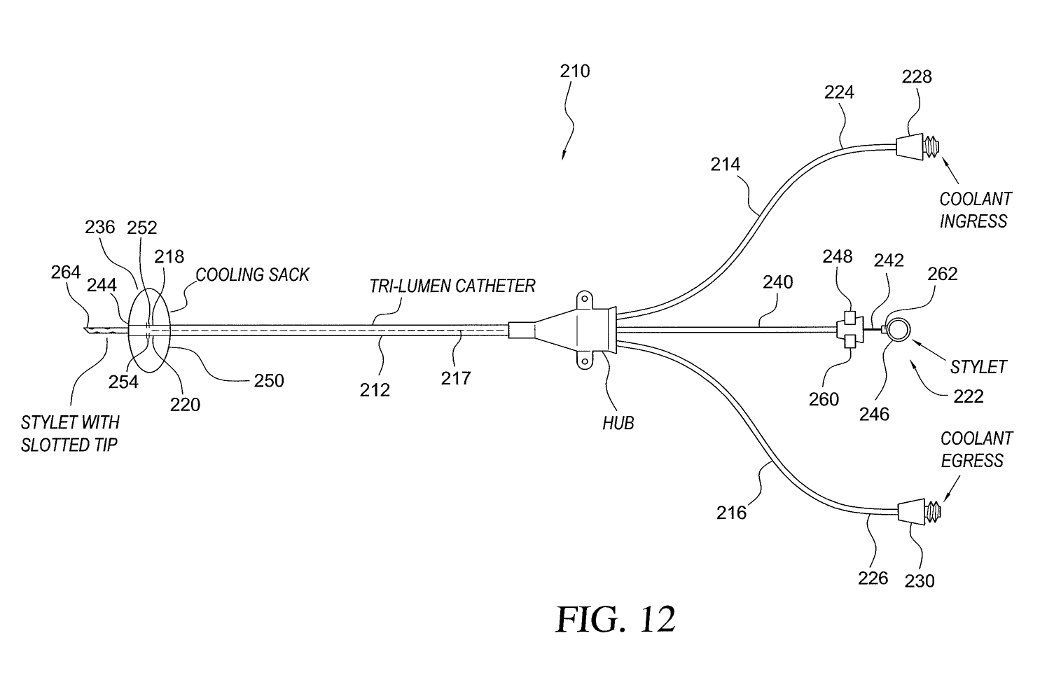

Referring now to FIG. 12, an alternate embodiment in accordance with the present invention is disclosed. The cooling catheter 210 of this embodiment is a tri-lumen polyurethane catheter. That is, the cooling catheter 210 is generally composed of a cylindrical, extruded tube 212 with three channels, that is, inlet and outlet channels 214, 216, providing for the flow of cooling fluid into and out of the cooling catheter 210, as well as a stylet channel 240 for the extension and retraction of a stylet 242 extending from the distal tip 244 of the cooling catheter 210.

More particularly, and in accordance with a preferred embodiment of the present invention, the cooling catheter 210 is approximately 33 cm long. The cooling catheter 210 has an outer diameter of approximately 3.3 mm, an inner diameter of approximately 2.7 mm and wall thickness of approximately 50 μm. The septum 217 separating the inlet and outlet channels 214, 216 and the stylet channel is approximately 50 μm thick.

The distal ends 218, 220 of the inlet and outlet channels 214, 216 formed within the cooling catheter 210 are in fluid communication so that a cooling fluid may be freely circulated within a closed loop extending through the cooling catheter 210. With this in mind a self-expanding, soft sack 250 is formed at the distal end 236 of the cooling catheter 210. In accordance with a preferred embodiment, the sack is made from a medical grade elastomeric polymer. The sack 250 is shaped and dimensioned such that when it is filled it takes the shape and size of the lateral ventricle 112 in which it is positioned in the manner discussed below in greater detail. The sack 250 is in fluid communication with both the inlet channel 214 and the outlet channel 216 via respective ports 252, 254 allowing for fluid communication between the sack 250 and the respective inlet and outlet channels 214, 216. As a result, the sack 250 is continually flushed with cooling fluid as the cooling fluid moves through the cooling catheter 210 of the present invention.

In practice, cooling fluid flows down the inlet channel 214, into the sack 250, and back up the outlet channel 216, providing for filling and expansion of the sack 250 along with cooling at the location of the sack 250 and along the entire length of the cooling catheter 210. At the proximal end 222 of the cooling catheter 210, the inlet and outlet channels 214, 216 split into individual tubes. The proximal ends 224, 226 of the respective channels 214, 216 are provided with a luer connection 230, 228 for fitting tubes (not shown) to supply (inlet) and remove (outlet) cooling fluid from the cooling catheter 210.

As briefly discussed above, a stylet 242 extends from the distal end 236. In particular, the distal tip 244 of the cooling catheter 210 is approximately 0.5 cm distal of the distal most portion of the sack 250.

As briefly mentioned above, the cooling catheter 210 is provided with a slotted stylet 242 that extends from the distal tip 244 of the cooling catheter 210 and may be selectively removed from the cooling catheter 210 as discussed below in greater detail. The stylet 242 includes a proximal end 246 accessible from the proximal end of the cooling catheter 210 for manipulation of the stylet 242 between its extended position and its withdrawn position. With this in mind, and considering the use of the stylet 242 as discussed below in greater detail, the cooling catheter 210 is provided with a selective frictional locking member 248 along the stylet channel 240 for maintaining the stylet 242 in a desired orientation until the cooling catheter is properly positioned at which time the stylet 242 may be removed. The stylet channel 240 is also provided with a valve member 260 selectively preventing the flow of fluid therethrough once the stylet 242 has been removed. In addition, the stylet 242 is provided with a stopcock valve 262 at its proximal end 246. The stopcock valve 262 permits the controlled flow of fluid into and out of the stylet 242.

With regard to the cooling system, the cooling systems described above for use in conjunction with the embodiment disclosed with reference to FIGS. 4 and 5 would certainly be appropriate for use in conjunction with the present embodiment.

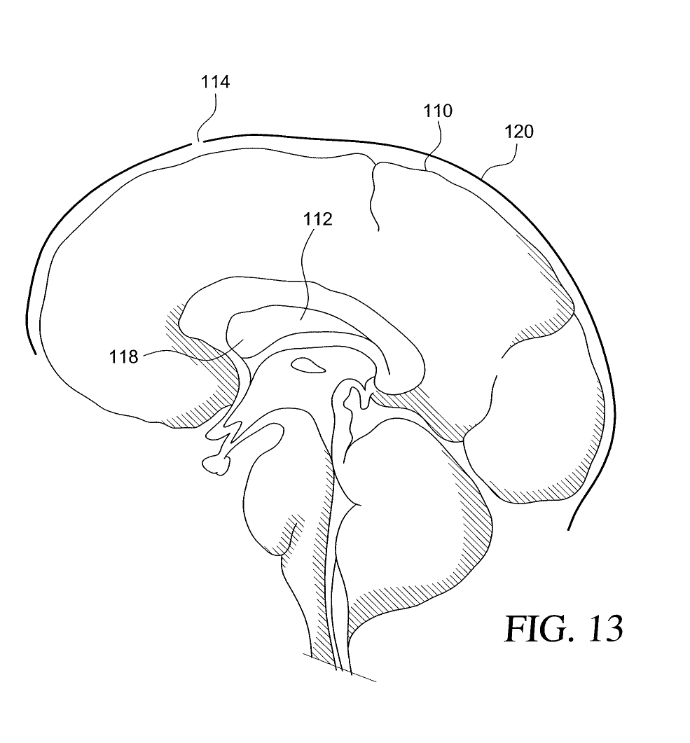

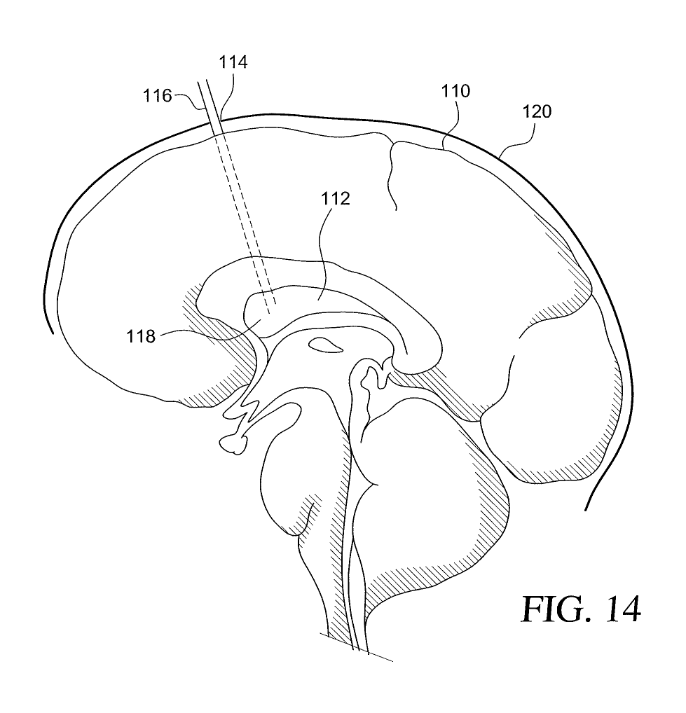

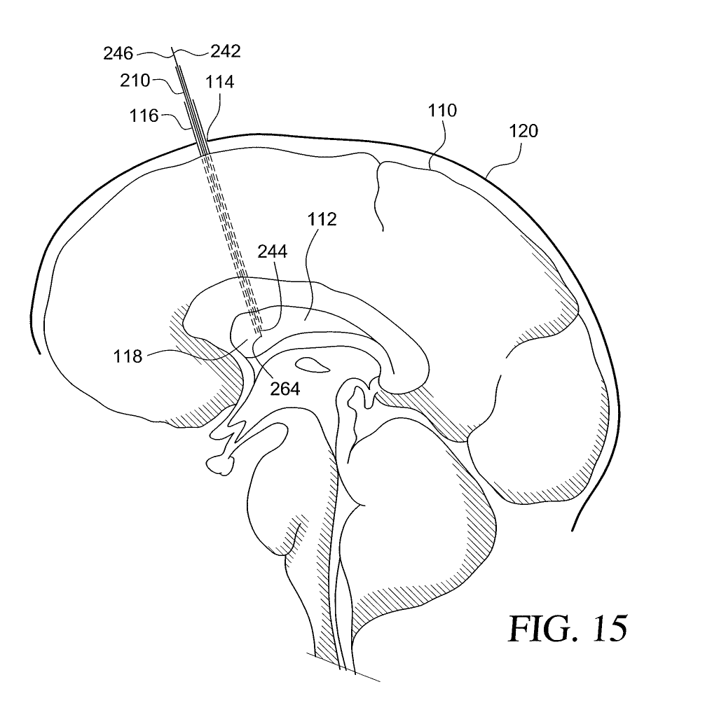

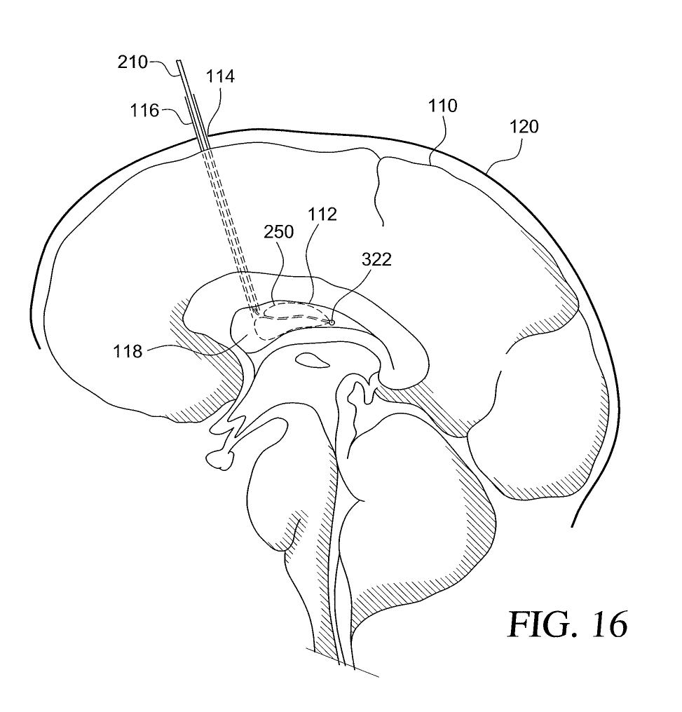

Referring to FIGS. 13 to 16, the present cooling catheter 210 is designed to provide hypothermic brain protection. Such brain protection would be provided in situations of cerebrovascular accident (for example, stroke) and traumatic brain injuries. In such situations, it is a standard neurosurgical practice to access one lateral ventricle 112 of the brain 110 via a burr hole 114 and a directed needle 116 puncture. As those skilled in the art will certainly appreciate, the lateral ventricles 112 form a portion of the ventricular system of the brain 110 and contain a reservoir of cerebral spinal fluid. In particular, the lateral ventricles 112 connect to the central third ventricle through the interventricular foramina of Monro.

In accordance with a preferred embodiment of the present invention, and with reference to FIGS. 13 to 16, a burr hole 114 is first formed in the skull 120 in accordance with traditional medical procedures those skilled in the art will certainly appreciate. The lateral ventricle 112 is then accessed via the burr hole 114 and the directed needle 116 puncture, the present cooling catheter 210, with the stylet 242 extended, is inserted through the needle 116 and into the ventricular cavity 118. Once the distal tip 244 of the cooling catheter 210 reaches the ventricular cavity 118, the cerebral spinal fluid will enter the distal end 264 of the stylet 242 and flow to and out of the proximal end 246 of the stylet 242. Once the flow is observed, the stopcock valve 262 is closed and the medical practitioner will know the sack 250 is properly positioned. The stylet 242 may then be removed from the cooling catheter 210 and the stylet channel 240 may be used as a ventricular drain if necessary.

For use in accordance with this procedure, the cooling catheter 210 is shaped and dimensioned such that the sack 250 may be positioned within the ventricular cavity 118 and then expanded to fill the ventricular cavity 118 when the cooling fluid is pumped therethrough. Once the cooling catheter 210 is properly positioned, cooling fluid is recirculated through the channels 214, 216 of the cooling catheter 210 as described above. This will cause the sack 250 to fill with cooling fluid, expand and fill the ventricular cavity 118. In general, and as discussed above with the spinal cord applications, the ventricular cavity 118 is preferably cooled to a temperature of between approximately 28° C. and approximately 34° C. and maintained at this temperature for a few hours to 1 to 3 days as required.

Improved functionality of the cooling catheter 210 in the performance of this procedure may be achieved by incorporating a monitor, for example, a pressure transducer 322, for measuring intracranial pressure.

While the preferred embodiments have been shown and described, it will be understood that there is no intent to limit the invention by such disclosure, but rather, is intended to cover all modifications and alternate constructions falling within the spirit and scope of the invention.

1. A method for cooling of a brain with localized hypothermia allowing for maintenance of core body temperature, comprising the following steps:
positioning a cooling catheter within a ventricular cavity of the brain, the cooling catheter including an inlet channel and an outlet channel providing for a closed flow of a cooling fluid into and out of the cooling catheter, a sack is formed at a distal end of the cooling catheter, the sack being in fluid communication with distal ends of the inlet channel and the outlet channel such that the sack is continually flushed with the cooling fluid as the cooling fluid flows into and out of the cooling catheter, wherein the sack, when filled, takes the shape and size of the ventricular cavity filling the ventricular cavity in which it is positioned;
cooling the cooling catheter and the ventricular cavity through the closed flow of the cooling fluid through the cooling catheter.

2. The method according to claim 1, wherein the cooling catheter is a tri-lumen catheter.

3. The method according to claim 1, wherein the cooling catheter is a tri-lumen catheter and, in addition to the inlet channel and the outlet channel, includes a stylet channel or fluid drainage channel.

4. The method according to claim 3, further including the step of drawing fluid from the ventricular cavity via the fluid drainage channel.

5. The method according to claim 1, wherein the sack is made from a medical grade elastomeric polymer.

6. The method according to claim 1, wherein the step of cooling includes the cooling fluid flowing down the inlet channel, into the sack, and back up the outlet channel, providing for filling and expansion of the sack along with cooling at a location of the sack.

7. The method according to claim 1, wherein the ventricular cavity is that of a lateral ventricle of the brain.

8. The method according to claim 1, wherein the step of positioning includes accessing the ventricular cavity via a burr hole.

9. The method according to claim 1, wherein the step of cooling includes cooling cerebral spinal fluid in the ventricular cavity to a temperature of between approximately 28° C. and approximately 34° C.

10. The method according to claim 1, wherein the step of cooling includes cooling for approximately several hours to 3 days.

11. The method according to claim 1, wherein the cooling catheter includes a monitor measuring intracranial pressure.

12. The method according to claim 1, wherein the cooling catheter includes a ventricular drain.
